(19) United States
(12) Patent Application Publication
(10) Pub. No.: US 2016/0143651 A1
(43) Pub. Date: May 26, 2016

(54) WINDOWED INSTRUMENT DRILL GUIDE AND CORRESPONDING FRICTION REDUCING INSTRUMENT

(71) Applicant: SMITH & NEPHEW, INC., Memphis, TN (US)

(72) Inventors: Timothy Young, Natick, MA (US); Amanda Simard, Hopkinton, MA (US); Richard M. Lunn, Kingston, MA (US); John Slusarz, Hopedale, MA (US); Paul A. Torrie, Marblehead, MA (US)

(21) Appl. No.: 14/946,568

(22) Filed: Nov. 19, 2015

Related U.S. Application Data
(60) Provisional application No. 62/081,614, filed on Nov. 19, 2014.

(51) Int. Cl. A61B 17/17

(52) U.S. Cl. CPC A61B 17/17

(57) ABSTRACT

In a first embodiment, an instrument guide includes an elongate guide body extending between a proximal end and a distal end, wherein a portion of the guide body extending proximally from the distal end is generally arcuate. The instrument guide further includes a rigid tip extending generally linearly from the distal end of the guide body. The rigid guide further includes a cannulation formed in the guide body and extending continuously from the proximal end of the guide body to a distal terminus of the tip. The cannulation can be approximately uniform in diameter. The instrument guide further includes a cut-out region, different from the guide body cannulation, extending along at least a portion of an inner radial surface of the arcuate body portion and intersecting the guide body cannulation.

### CROSS-REFERENCE TO RELATED APPLICATIONS

The subject application relates to and claims priority to U.S. Provisional Application Ser. No. 62/081,614 filed on Nov. 19, 2014, the contents of which are hereby incorporated by reference.

### BACKGROUND

The subject application relates to surgical instruments and more particularly to apparatus, systems and methods for facilitating/guiding insertion of surgical instruments along a curved trajectory.

Insertion of surgical instruments, such instruments for manipulating, imaging, or otherwise interacting with a patient's internal anatomy is often accomplished using a guide channel or cannula. In some instances, depending on the particular surgical procedure or to ensure an angled delivery of the surgical instrument, it may be desirable to have an insertion path that bends or curves. Thus, the guide channel or cannula may typically include a bend or curvature to facilitate such a bent or curved entry path.

Problems arise, however, when attempting to insert rigid sections of instrumentation (drill bits, anchors, etc.) through a bent/curved region of the guide. In such instances, the bend/curve in the guide either needs to be very gradual, or there needs to be a large amount of clearance between the inner diameter of the guide and outer diameter of a rigid section being passed through.

If the bend/curve is not gradual and the clearance is too tight, the rigid section may become “stuck” (e.g., as shown in FIG. 1-1), which may damage the instrument or prevent insertion thereof. Conversely, if the rigid section is too loose within the inner diameter of the guide, the rigid section may not come out concentric with the outer and inner diameters of the guide. Thus, it is favorable for a rigid section to have a tight/snug fit within the distal tip of the guide, thereby ensuring that the rigid section exits the guide concentric and in-line with to the outer and inner diameter of the distal end of the guide. Having the rigid section come out concentric and in-line with the distal end of the guide better allows the surgeon to access a position of the surgical instrument once extended from the distal end of the guide.

Thus, improved apparatus, systems and methods for facilitating/guiding insertion of surgical instruments along a curved trajectory are needed. These and other needs are addressed by way of the present disclosure.

### SUMMARY OF THE DISCLOSURE

Exemplary embodiments of an instrument guide are disclosed herein the guide including an elongate guide body extending between a proximal end and a distal end, wherein a portion of the guide body extending proximally from the distal end is generally arcuate. The rigid guide further includes a cannulation formed in the guide body and extending continuously from the proximal end of the guide body to a distal terminus of the tip. The cannulation can be approximately uniform in diameter. The instrument guide further includes a cut-out region, different from the guide body cannulation, extending along at least a portion of an inner radial surface of the arcuate body portion and intersecting the guide body cannulation.

In example embodiments, the instrument guide further includes a rigid tip extending generally linearly from the distal end of the guide body.

In embodiments, the instrument guide can be a drill guide or an scope guide, for example.

in example embodiments, the instrument guide the cut-out region may be a rectangular slot.

In example embodiments, the cut-out region may be configured to enable a portion of a rigid instrument to extend or protrude beyond an inner diameter of guide as defined by the cannulation while traversing the arcuate portion of guide. In some embodiments, the cut-out region may constrain the rigid instrument from protruding or extending beyond the outer diameter of the guide.

In example embodiments, the cut-out region may extend all the way between an inner diameter of the guide as defined by the cannulation and an outer diameter of the guide. In other embodiments, the cut-out region may be covered or extend only part of the way between an inner diameter of the guide as defined by the cannulation and an outer diameter of the guide.

In example embodiments, the cut-out region is specifically configured to provide a selected tolerance range for a pre-determined rigid instrument based a geometry of a rigid portion of the instrument, a curvature of the arcuate portion of guide and a cannulation diameter of the instrument.

In example embodiments, the cut-out region may be specifically configured to provide a selected tolerance range for a pre-determined rigid instrument based a geometry of a rigid portion of the instrument, a curvature of the arcuate portion of guide and the cannulation, e.g., cannulation diameter, may be specifically configured to provide close enough tolerance to ensure that the surgical instrument remains concentric with a distal end of the guide and/or in-line with a longitudinal axis of the guide at a distal end thereof, when the surgical instrument is extended from the distal end of the guide.

In example embodiments, the instrument guide may be included as part of a surgical kit further including a sleeve for a surgical instrument the sleeve having an elongate inner member extending between a proximal end and a distal end, the distal end adapted to engage a rigid instrument; and a tubular outer member extending between a proximal end and a distal end, the outer member extending through the sidewalls thereof; wherein the inner member is positioned within a cannulation defined by the outer member such that the outer surface of the inner member contacts the inner surface of the outer member; and wherein the inner member is secured to the outer member such that the outer member is axially constrained with respect to the inner member. In some embodiments the kit may further include a surgical instrument having a rigid portion, wherein the elongate inner member of the sleeve is engaged to or forms part of the surgical instrument.

In example embodiments, the instrument guide may be included as part of a surgical kit further including a surgical instrument having a rigid portion.

Also disclosed herein are exemplary embodiments of an instrument sleeve, the sleeve including an elongate inner member extending between a proximal end and a distal end, the distal end adapted to engage a rigid instrument. The instrument sleeve further includes a tubular outer member extending between a proximal end and a distal end, the outer member extending through the sidewalls thereof. The inner member is positioned within a cannulation defined by the outer member such that the outer surface of the inner member contacts the inner surface of the outer member. The inner member can be secured to the outer member such that the outer member is axially constrained with respect to the inner member.

In example embodiments, the outer member may be advantageously configured to provide for a narrow tolerance with respect to a cannulation of a guide shaft.

In example embodiments, the outer member can have a plurality of discontinuous helical cuts.

In some embodiments, the outer member may be configured to rotate with the engaged rigid instrument. In further embodiments, the outer member may be rotationally keyed to a curved lumen of a bent shaft.

In example embodiments, the inner member may be a sheath structure configured to fit over and thereby engage the rigid instrument.

Also disclosed herein are example embodiments of a surgical instrument, the instrument including a body having a first diameter and including a rigid member and a flexible bearing element having a second diameter greater than the first diameter, wherein the bearing element is effective to axially constrain the body.

In example embodiments, the body is a drill tip.

In further embodiments, the instrument may include a shaft element attached relative to the body. In some embodiments, the bearing element is over molded relative to the shaft element. The shaft element may be constructed of a flexible or sepal-flexible wire or other material. In some embodiments, the shaft element may have a smaller diameter than the body.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments of the present disclosure.

FIG. 1-1 is a diagram illustrating a cut-away of an instrument guide with a rigid instrument navigating therein;

FIG. 1-2 is an example embodiment of the present instrument guide;

FIG. 1-3 shows an isometric view of an example embodiment of the present instrument guide;

FIGS. 1-4A through 1-4F show example embodiments of incremental movements of the rigid instrument through the instrument guide;

FIG. 1-5 illustrates a curved instrument guide on an acetabular rim;

FIG. 1-6 shows a windowed guide of the present disclosure on an acetabular rim;

FIG. 1-7 shows the windowed guide of the present disclosure and the curved windowless instrument guide of other instrument guides;

FIG. 2-1 is a diagram of an example embodiment of flexible plastic sleeve technology;

FIG. 2-2 is a diagram of an example embodiment of a cross section of flexible plastic sleeve technology; and

FIG. 2-3 is a diagram of an example embodiment of a prototype drill. A singular plastic bearing is overmolded onto 0.050 NiTi (Nickel Titanium) wire.

### DETAILED DESCRIPTION

A description of example embodiments of the apparatus, systems and methods disclosure herein follows:

As noted above, many instrument guides utilize a bend at a distal end of the instrument guide. An instrument guide having such a bend allows surgeons to obtain a more favorable trajectory when drilling and placing anchors into bone, or performing other activities with the instrument guide (it may be desirable to have an insertion path that bends or curves to ensure an angle of insertion or to work around given anatomical constraints). Thus, the guide channel or cannula may typically include a bend or curvature to facilitate such a bent or curved entry path Apparatus, systems and methods are presented herein for facilitating/guiding insertion of surgical instruments along a curved trajectory.

Furthermore, as noted above, in conventional instrument guides, the bend/curve in the guide either needs to be very gradual, or there needs to be a large amount of clearance between. the inner diameter of the guide and outer diameter of a rigid section being passed through. A slight bend, however, may not be desirable/workable for a given procedure. Moreover, increasing the clearance of the cannula can have a negative impact on the exit trajectory of a surgical instrument. The apparatus, systems and methods of the present disclosure address these issues by enabling steeper bends in the guide without forgoing a narrow tolerance that ensures that a surgical instrument exit the guide concentric with to the outer and inner diameter of the distal end of the guide and in-line with a longitudinal axis thereof As noted above, having the rigid section come out concentric and in-line with the distal end of the guide better allows the surgeon to access a position of the surgical instrument once extended from the distal end of the guide.

Presently available instrument guides do not allow a rigid instrument to consistently emerge from the distal end having a concentric trajectory. In addition, some instrument guides are limited by using gradual curves to allow the rigid instrument to pass through the bend without getting stuck in the instrument guide. In other instrument guides, a separate ‘guide tip’ welded to the distal end of the instrument guide, which has a smaller inner diameter than the rest of the instrument guide, allows the fit between the rigid instrument outer diameter and distal guide inner diameter to be tighter and also allowing the rigid instrument to navigate the curve in a “loose fitting” bend.

In a first embodiment of the disclosure, an instrument guide with a bend has a window cutout to allow rigid instruments to navigate the bend and emerge concentrically from the distal end of the instrument guide. The window cutout allows the rigid instrument to navigate the bend without sacrificing the concentricity of the rigid instrument relative to the instrument guide by maintaining a normal fit on the rigid part and without increasing the overall outer diameter of the guide. Additional navigation area for the rigid instrument is obtained by removing material from the inside radius wall of the tube, however, the fit of the rigid instrument is otherwise maintained.

In a second embodiment of the disclosure, a friction reducing surface can be placed on the rigid instrument. Reduced friction helps the rigid instrument pass through the bend of the instrument guide.

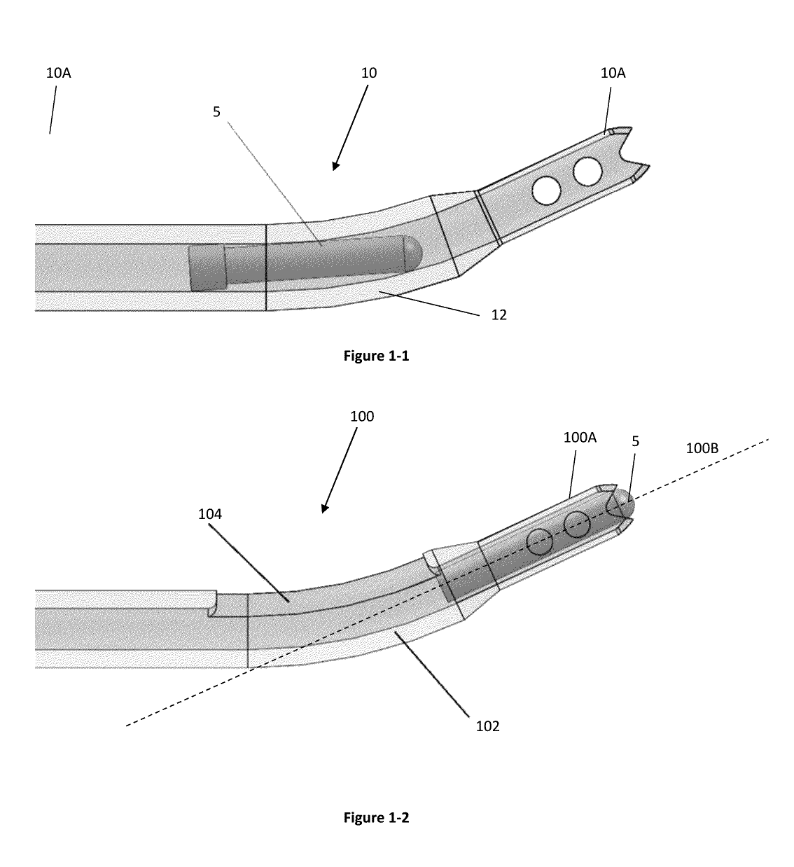

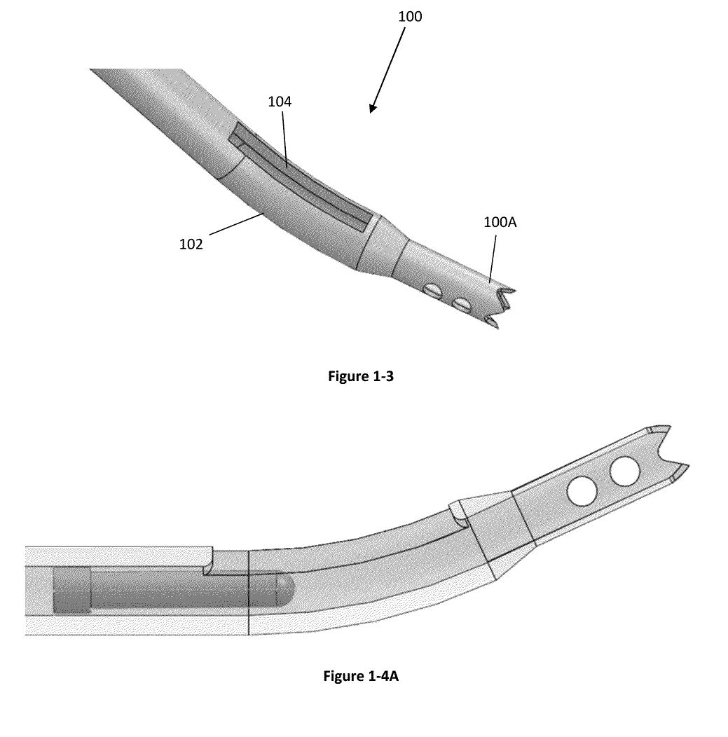

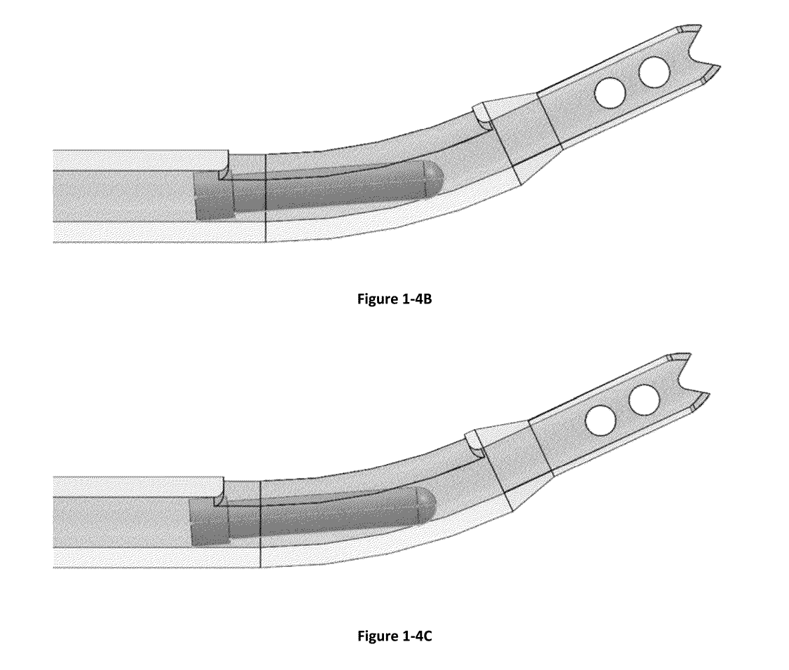

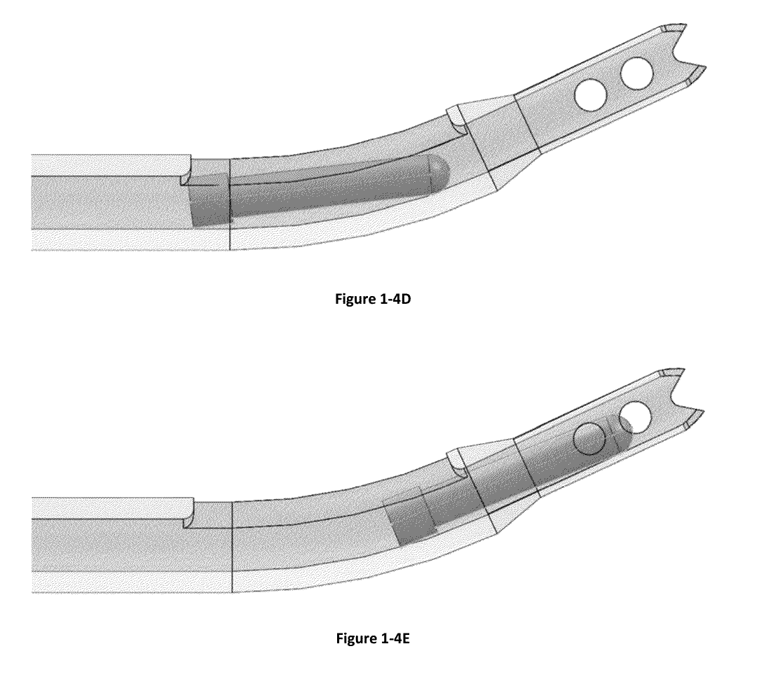

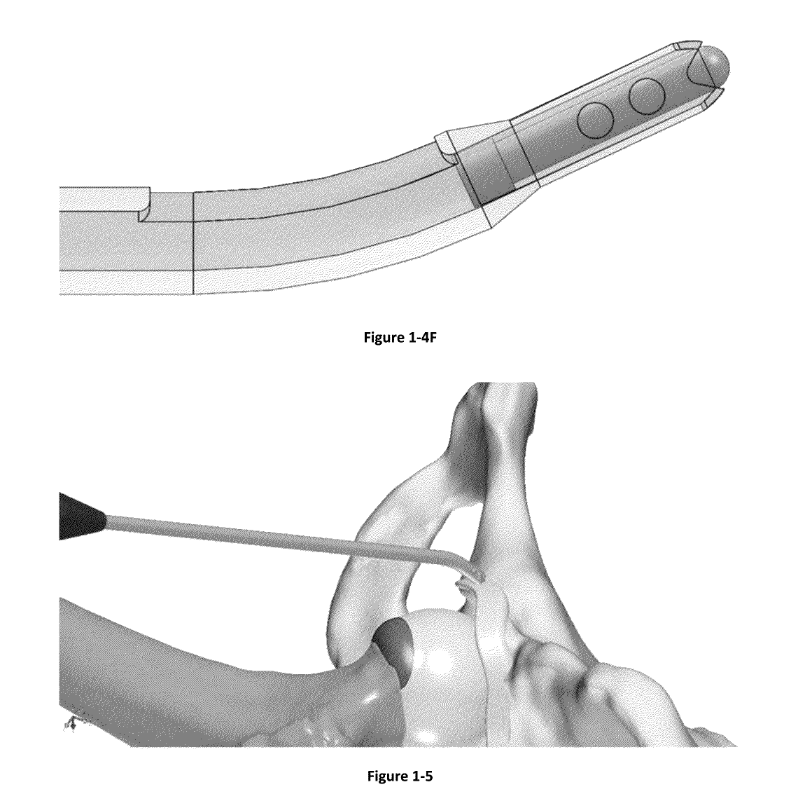

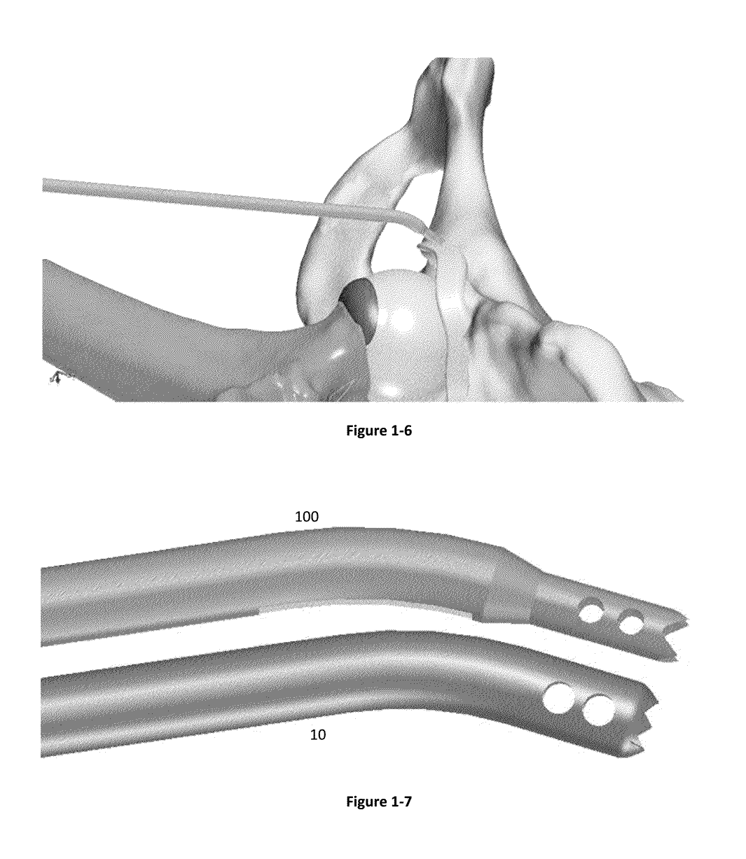

FIG. 1-1 is a diagram illustrating a cut-away view of a conventional instrument guide 10 with a rigid instrument 5 navigating therein. The guide of FIG. 1-1 is curved (curve 12), however, the rigid instrument 5 emerging from the distal tip I OA may not be concentric with the instrument guide 10. Further, the rigid instrument 5 could become stuck. in the guide 10 if the fit through the curve 12 is too tight. As depicted, the rigid instrument 5 cannot navigate the curve 12 of the instrument guide 10 because the fit is too tight. That is, the inner diameter of the guide 10 is too small to allow the rigid instrument 5 to traverse the curve 12 because the rigid instrument 5 cannot bend along with the guide 10.

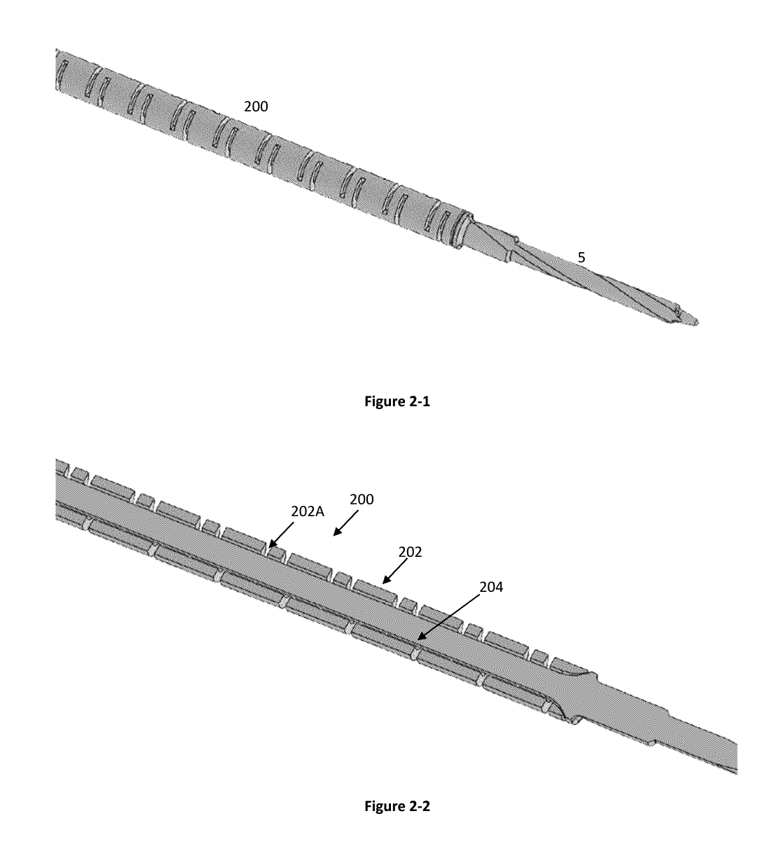

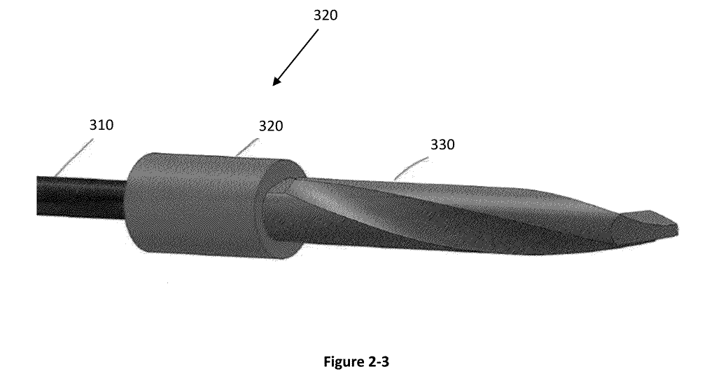

With reference now to FIG. 1-2, a cut-away view of an example embodiment of an improved instrument guide 100 is depicted, according to the present disclosure. Similar to the conventional instrument guide 10 of FIG. 1-1, the instrument guide 100 of FIG. 1-2 includes a bend 102 that allows surgeons to better position an angle of a surgical instrument during surgery. The instrument guide 100 further includes a window cutout 104. The window cutout 104 advantageously allows the rigid instrument S to traverse the bend 102 without sacrificing narrow tolerances of the inner diameter of the guide 100. Thus, the rigid instrument 5 can remain within the constraints of (e.g., tight or flush) the inner diameter of the guide 100 even as it traverses through the bend 102, but has flexibility to move unimpeded due to the window 104. This configuration advantageously enables the rigid instrument 5 to emerge concentric and in-line with the guide 100. That is, the rigid instrument 5 will have the same longitudinal axis 100B of the guide 100 at a distal end 100A.

FIG. 1-3 shows an isometric view of the same example embodiment of an improved instrument guide depicted in FIG. 1-2. As shown, the window cutout 104 of the surgical instrument 100 of FIGS. 1-2 and 1-3 is a rectangular slot positioned along a concave (inner curve) side of the guide 100. A person of ordinary skill in the art will appreciate that any number of different geometric configurations of the window 104 may be utilized without departing from the scope of the present disclosure. For example, in some embodiments the window cutout 104 may be an oval shaped slot, a tapered slot, or some other geometric configuration. In general, the window cutout 104 is configured to enable a portion of a rigid instrument 5 to extend/protrude beyond the inner diameter of the guide 100 while traversing the bend 102. In some embodiments, the window cutout 104 may further be configured to enable a portion of the rigid instrument 5 to protrude/extend beyond an outer diameter of the guide 100. Thus, in some embodiments, the window cutout 104 may extend all the way between the inner and outer diameters of the guide 100. In other embodiments, the window cutout 104 may extend only part of the way between the inner and outer diameters of the guide 100. Thus, in some embodiments, the window cutout 104 may constrain the rigid instrument 5 from protruding/extending beyond the outer diameter of the guide 100. In example embodiments the window cutout 104 may be configured to provide for a specific tolerance for a given rigid instrument 5 as determined by the length and diameter of the rigid portion of the instrument 5, the curvature of the bend 102 and/or the inner and/or outer diameters of the guide 100.

FIGS. 1-4A through 1-4F show an example embodiment of incremental movements of the rigid instrument through the instrument guide. In FIG. 1-4A, the rigid instrument is shown before it passes through the bend of the drill bit. In FIG. 1-4B, the rigid instrument is shown within the bend and a portion extending/protruding through the window cutout beyond the inner diameter of the guide, In FIG. 1-4C, the rigid instrument is shown even further within the bend and having a greater portion extending/protruding through the window cutout beyond the inner diameter of the guide. In FIG. 14D, the rigid instrument is shown entirely within the bend, partially using the space cut out by the window. In FIG. 14E, the rigid instrument is shown exiting the bend and beginning to enter and align with the distal tip. The portion of the rigid instrument within the bend is still occupying space in the window where the outer portion of the bend would have been. In FIG. 1-4F, the rigid instrument is shown to be entirely in the distal end having successfully traversed the bend using the window cutout and is concentric with the instrument guide.

FIG. 1-5 illustrates a conventional curved instrument guide on an acetabular rim. In contrast, FIG. 1-6 shows a curved instrument having window guide such as disclosed herein on an acetabular rim. Further contrasting the technology, FIG. 1-7 shows the windowed instrument guide 100 of the present disclosure and the curved windowless instrument guide 10 of a conventional guide. The instrument guide having embodiments of the windowed portion allows for the same bend in the instrument guide with the rigid instrument emerging from the instrument guide concentric. A distal piece also does not need to be welded to the end of the instrument guide, which is how many existing guides are manufactured. Instead, the rigid instrument can emerge from the distal end after traversing the instrument guide and be used by the surgeon once emerged.

In other embodiments, the window can have a variety of window cut lengths, widths, geometries, and guide bends. The guide bend can have a variety of uniform or nonuniform curvatures. The instrument guide can also utilize a thin plastic or metal sheet that covers the window to prevent soft tissue from being entrapped in the instrument guide through the window. In embodiments, the thin plastic or metal sheet can be pliable or bendable so that the rigid instrument can pass through the window and bend the sheet if necessary as it passes through. In example embodiments, the width of the window is less than the interior diameter of the guide and/or less than the diameter of the surgical instrument.

The present instrument guide is a guide design allowing a rigid instrument to navigate through a curve without sacrificing bend radii or implementing a large inner diameter. The rigid instrument at the distal tip of the guide can be the same length or slightly longer than the rigid instrument of the navigating instrumentation allowing the drill and anchor to exit the guide concentric to the distal guide tip. The present design can be a single tube design with no need for a welded distal guide tip.

In a second embodiment, a flexible plastic surface acts as both a wear surface and bearing sleeve for curved instrumentation, As described above, curved suture anchor systems utilize a bend at the end of the instrument guide to allow surgeons to obtain a more favorable trajectory when drilling and placing anchors into bone. A problem arises when attempting to cleanly allow instrumentation. passage through the bend in the instrument guide.

One common issue When getting instrumentation around a bend is surface to surface contact, which results in friction. The surface to surface contact causes increased difficulty to advance instruments and potential damage. Over time, wear from surface to surface contact is also a problem for a reusable device.

Another issue when getting instrumentation around the bend is a potential for necessary loosened tolerances between the guide and the instrumentation. Loose tolerances can result in the rigid instrument at the end of the curved device (drill tip or anchor), which is passing through the lumen, to exit the lumen in a “non-concentric” trajectory. This is unfavorable when drilling in bone and inserting suture anchors because it does not allow the surgeon to accurately predict where he is drilling/inserting within the bone.

Therefore, obtaining a concentric exit from the guide is desired from the time which the tip of advancing instrumentation (drill or inserter anchor) exits the distal lumen of the guide until the appropriate advancement depth is achieved. Obtaining a concentric trajectory after the instrumentation is at its full advancement depth is of no benefit. Concentric trajectory after final advancement does not change the inline path which the drill or anchor has taken. Rather, it only exerts a radial force on the surrounding bone.

Present curved guides utilized technology such as helical hollow strand (HHS) coils, 180 degree and/or 120 degree wire electrical discharge machining (EDM) cuts, puzzle cut technology (e.g., Avalign), springs, thin or “necked down” rods of metal, plastic, Nitinol, and joined outer rings to center the rigid instrumentation. Each of these technologies has a downside, whether it is cost, friction, or strength.

In a further embodiment of the disclosure, a sleeve is presented which advantageously reduces friction on a rigid instrument to allow easier passage through a curved instrument guide, e.g., an improved instrument guide with a cutout window as described herein.

With reference now to FIG. 2-1, a diagram of an example embodiment of a flexible sleeve 200 for a surgical instrument 5 is depicted. In an embodiment, the flexible sleeve 200 can be plastic. In other embodiments, however, the flexible sleeve 200 can be non-plastic and made out of a material such as brass, for example. FIG. 2-2 is a cut-away view of the flexible sleeve 200 of FIG. 2-1. The sleeve technology includes two parts: an outer material 202 and inner material 204. The outer material 202 of the sleeve 200 can be comprised of a plastic surface that has helical (e.g., 120 degree) rectangular cuts 202A. These cuts 202A provide room for the technology to bend when in a tightly toleranced and curved lumen. The plastic can rub against the curved lumen while acting as a wear and bearing surface to control the instrumentation. Because the plastic is a flexible and somewhat malleable material, tight tolerances of the outer plastic material and curved lumen which the technology is constrained in can be achieved. This outer material 202 goes over and is constrained to a tight fitting inner material 204. The outer and tight fitting material allows for the rigid instrument at the end of the flexible technology to come out concentric with the distal inner diameter of the lumen (in the case of arthroscopy, an instrument guide). The plastic material can be constrained to the inner material by any number of methods, the primary method being overmolding.

The inner material 204 of the sleeve 200 may be, for example, a rod composed of another material (stainless steel, Nitinol) acting as the torsional & axial strength bearing member or an inner sheath type structure/material, This inner material 204 is joined to the outer material 104 by a method (e.g., over-molding, snap fit, threading [e.g., in the case of a helical wire embodiment which can be screwed onto the shaft], adhesive, etc.) which maintains concentricity of the outer 204 material with respect to the inner material 202. In addition, the bell shaped features on the shaft constrain the axial movement of the outer material.

In another embodiment, however, the outer material 202 can free float on the inner material 204 as long as it is constrained axially. The outer material 202 acts as a radial spacer. In embodiments, the outer material may also rotate with the drill (e.g., through the snap threading) or may be rotationally keyed to the curved lumen of the bent shaft, which moves the sliding surface closer to the shaft's center of rotation. This reduces surface speed and therefore reduces wear on the device.

The combination of these two outer and inner materials allow for a technology that can navigate a tightly toleranced and curved lumen with minimal resistance, while still providing adequate torsional and axial strength. All this is achieved with the rigid instrument of the instrumentation emerging concentric (or ‘on-center’) to the inner diameter of the distal end of the guide.

One embodiment of the present disclosure is a helical plastic tubing section which has a slit placed lengthwise. The tubing is attached to the inner material by plying it open and snapping into place, or by threading, as described above.

FIG. 2-3 is a diagram of an example embodiment of a prototype drill 300. A singular plastic bearing 320 is overmolded onto base shall 310 (0.050 NiTi [Nickel Titanium] wire), A drill tip 330 is attached to the end. Other embodiments of this prototype drill can include adding additional plastic “rings” more proximal on the wire. The bearing 320 may be configured to include similar elements and aspects as described above with respect to the sleeve technology.

In another embodiment, the inner material can be ‘necked down’ to constrain the axial movement of the outer material. In yet another embodiment, the outer material can have various degrees and lengths of rotational/axial spacing configurations respectively. In another embodiment, the outer material can have a helical nature that supports flexibility and rotation.

In another embodiment, the outer material can simply be two to three proximal and distal plastic bearing surfaces that have open space between them (with the exception of the connecting inner material) which guide the flexing section around the bend. In another embodiment, the outer material can be constrained axial with respect to the inner material by any number of methods or combinations of these methods: (1) Necked down inner material (e.g., FIGS. 2-1 & 202), (2) Overmolded outer material, (3) Shrunk down tubing (shrink tube), or (4) “Snapped on” over the inner material.

In another embodiment, the outer tube may or may not be constrained rotationally with respect to the inner material, In another embodiment, various thickness ratios between the outer and inner materials.

In yet another embodiment, the technology can be applied to drills, inserters, obturators or any other device which must be passed down a curved or bent passage.

The embodiments of the present disclosure provide a relatively inexpensive solution to the problems in the art compared to other technologies such as puzzle cut. Further, the forgiving outer material allows the technology to be tightly toleranced and/or fit within the curved lumen. The inner material allows for axial and torsional strength of the technology while still permitting bend navigation. The combination of these two outer and inner materials allow for a technology which can navigate a tightly toleranced and curved lumen with minimal resistance, while still providing adequate torsional and axial strength. Minimal performance sacrifices are needed in order to obtain desired benefits.

The tight tolerance allows the rigid instrument at the end of the flexible segment to emerge concentric (“on center”) to the inner diameter of the guide lumen.

While this apparatus, systems and methods of the present disclosure have been particularly shown and described with references to example embodiments thereof, it will be understood by those skilled in the art that various changes in form and details may be made therein without departing from the scope of the present disclosure.

What is claimed is:

1. An instrument guide, comprising:
an elongate guide body extending between a proximal end and a distal end, wherein a portion of the guide body extending proximally from the distal end is generally arcuate;
a cannulation formed in the guide body and extending continuously from the proximal end of the guide body to a distal terminus of the tip, wherein the cannulation is approximately uniform in diameter; and
a cut-out region, different from the guide body cannulation, extending along at least a portion of an inner radial surface of the arcuate body portion and intersecting the guide body cannulation.

2. The instrument guide of claim 1, further comprising a rigid tip extending generally linearly from the distal end of the guide body.

3. The instrument guide of claim 1, wherein the cut-out region is a rectangular slot.

4. The instrument guide of claim 1, wherein the cut-out region is configured to enable a portion of a rigid instrument to extend or protrude beyond an inner diameter of guide as defined by the cannulation while traversing the arcuate portion of guide.

5. The instrument guide of claim 4, wherein the cut-out region constrains the rigid instrument from protruding or extending beyond the outer diameter of the guide.

6. The instrument guide of claim 1, wherein the cut-out region extends all the way between an inner diameter of the guide as defined by the cannulation and an outer diameter of the guide.

7. The instrument guide of claim 1, wherein the cut-out region is covered or extends only part of the way between an inner diameter of the guide as defined by the cannulation and an outer diameter of the guide.

8. The instrument guide of claim 1, wherein the cut-out region is specifically configured to provide a selected tolerance range for a pre-determined rigid instrument based a geometry of a rigid portion of the instrument, a curvature of the arcuate portion of guide and a cannulation diameter of the instrument.

9. The instrument guide of claim 1, wherein the cut-out region is configured to provide sufficient clearance for a rigid portion of a surgical instrument to traverse the arcuate portion of guide via the cannulation and wherein the cannulation is configured to provide close enough tolerance to ensure that the surgical instrument remains concentric with a distal end of the guide when extended therefrom.

10. The instrument guide of claim 1, wherein the cut-out region is configured to provide sufficient clearance for a rigid portion of a surgical instrument to traverse the arcuate portion of guide via the cannulation and wherein the cannulation is configured to provide close enough tolerance to ensure that the surgical instrument remains in-line with a longitudinal axis of the cannulation at a distal end of the guide when the surgical instrument is extended from the distal end of the guide.

11. A surgical kit comprising the instrument guide of claim 1 and an instrument sleeve including:
an elongate inner member extending between a proximal end and a distal end, the distal end adapted to engage a rigid instrument; and
a tubular outer member extending between a proximal end and a distal end, the outer member extending through the sidewalk thereof;
wherein the inner member is positioned within a cannulation defined by the outer member such that the outer surface of the inner member contacts the inner surface of the outer member; and
wherein the inner member is secured to the outer member such that the outer member is axially constrained with respect to the inner member.

12. The surgical kit of claim 11 further comprising a surgical instrument having a rigid portion, wherein the elongate inner member is engaged to the surgical instrument.

13. A surgical kit comprising the instrument guide of claim 1 and a surgical instrument having a rigid portion.

14. An instrument sleeve, comprising:
an elongate inner member extending between a proximal end and a distal end, the distal end adapted to engage a rigid instrument; and
a tubular outer member extending between a proximal end and a distal end, the outer member extending through the sidewalls thereof;
wherein the inner member is positioned within a cannulation defined by the outer member such that the outer surface of the inner member contacts the inner surface of the outer member; and
wherein the inner member is secured to the outer member such that the outer member is axially constrained with respect to the inner member.

15. The instrument sleeve of claim 14, wherein the outer member has a plurality of discontinuous helical cuts.

16. The instrument sleeve of claim 14, wherein the outer member is configured to rotate with the engaged rigid instrument

17. The instrument sleeve of claim 14, wherein the outer member is rotationally keyed to a curved lumen of a bent shaft.

18. The instrument sleeve of claim 14, wherein the inner member is a sheath structure configured to fit over and thereby engage the rigid instrument.

19. The instrument sleeve of claim 14, wherein the outer member is configured to provide for a narrow tolerance with respect to a cannulation of a guide shaft.

20. A surgical instrument comprising a body having a first diameter and including a rigid member and a flexible bearing element having a second diameter greater than the first diameter, wherein the bearing element is effective to axially constrain the body.

21. The surgical instrument of claim 20, wherein the body is a drill tip.

22. The surgical instrument of claim 20, further comprising a shaft element attached relative to the body.

23. The surgical instrument of claim 22, wherein the bearing element is over molded relative to the shaft element.

24. The surgical instrument of claim 22, wherein the shaft element is comprised of a flexible or semi-flexible wire or other material.

25. The surgical instrument of claim 22, wherein the shaft element has a smaller diameter than the body.
